(19) United States
(12) Patent Application Publication
(10) Pub. No.: US 2014/0179012 A1
(43) Pub. Date: Jun. 26, 2014

(54) METHOD OF ANALYZING A BLOOD SAMPLE

(71) Applicant: GLUCOME LTD., YARKONA (IL)

(72) Inventors: DOV MORAN, KFAR SABA (IL); YIFTAH BEN AHARON, MOSHAV BAKOA (IL)

(21) Appl. No.: 14/133,702

(22) Filed: Dec. 19, 2013

Related U.S. Application Data
(60) Provisional application No. 61/739,741, filed on Dec. 20, 2012.

(51) Int. Cl. G01N 33/66; G01N 27/327

(52) U.S. Cl. CPC G01N 33/66; G01N 27/3275
USPC 436/95; 205/782; 204/403.01; 422/82.02

(57) ABSTRACT

A method of analyzing a blood sample is provided. The method comprises providing a system configured to perform an analysis of a glucose level of the blood sample disposed on a test medium, wherein the test medium is one of a quantity of test media in a user's supply, monitoring, by the system, the number of test media of the supply used in performing the analysis, determining, by the system, that a threshold value of test media has been reached, the threshold value being less than the quantity, and performing, by the system, once the threshold value has been reached, an ordering procedure.

### CROSS-REFERENCE TO RELATED APPLICATIONS

This application claims the benefit of priority from U.S. Provisional Patent Application No. 61/739,741, filed Dec. 20, 2012, the contents of which are incorporated herein by reference in their entirety.

### FIELD OF THE INVENTION

The disclosure herein relates to the methods and systems for analyzing the glucose level of a blood sample. In particular, it relates to methods and systems for monitoring a supply of test media and facilitating ordering replacement test media.

### BACKGROUND OF THE INVENTION

Diabetes is a metabolic disease characterized by high blood sugar, also called glucose, resulting from disruption in production of, or lack of proper response to, insulin, a hormone central to regulating carbohydrate and fat metabolism.

More than 18.2 million people (or 6.3 percent of the population) in the United States suffer from diabetes. Diabetes can cause serious health complications including heart disease, blindness, kidney failure, and lower-extremity amputations. These complications may be avoided through effective and efficient balance of sugar levels. The glucometers (also called a glucose meter) is one tool for reaching and maintaining an optimal balance of blood sugar.

Many glucometers use an electrochemical method, based on test media such as test strips. Test strips are a consumable element containing chemicals that react with glucose in a drop of blood used for each measurement. The test media are typically single-use elements which are sold in packages which must be replaced once they are all used.

### SUMMARY OF THE INVENTION

According to one aspect of the presently disclosed subject matter, there is provided a method of analyzing a blood sample, the method comprising:

providing a system configured to perform an analysis of a glucose level of the blood sample disposed on a test medium, wherein the test medium is one of a quantity of test media in a user's supply;

monitoring, by the system, the number of test media of the supply used in performing the analysis;

determining, by the system, that a threshold value of test media has been reached, the threshold value being less than the quantity; and

performing, by the system, once the threshold value has been reached, an ordering procedure.

The system may comprise a glucometer configured to perform the analysis and transmit data, and a remote computing device configured to receive a transmission from the glucometer.

The remote computing device may be configured to communicate with an external network. For example, it may be selected from the group including a mobile phone (e.g., built on a mobile operating system) and a tablet computer.

The monitoring may comprise receiving an indication of the quantity of the user's supply of test media. The indication may be at least partially based on information regarding a previous ordering procedure.

The monitoring may comprise one (or both) of:

decrementing a value corresponding to the number of test media remaining in the user's supply when one or more test media is used; and

incrementing a value corresponding to the number of test media used from the user's supply when one or more test media is used.

The expected daily usage of test media may be at least partially based on at least one or more of:

expected daily usage of test media from the user's supply;

usage history thereof; and

expected shipping time of a new supply of test media.

The ordering procedure may comprise at least one or more of:

the system automatically ordering test media;

the system alerting a user to place an order; and

the system presenting information necessary to order test media to a user, wherein the method may further comprise the system ordering test media based on the information presented upon approval by a user.

The user's supply may comprise a single package of test media. It may comprise more than one package of test media.

According to another aspect of the presently disclosed subject matter, there is provided a system configured to perform an analysis of a glucose level of a blood sample disposed on a test medium, wherein the test medium is one of a quantity of test media in a user's supply, the system comprising:

a glucometer configured to perform the analysis and to transmit data; and

a remote computing device configured to receive a transmission from the glucometer and to communicate with an external network;

wherein the system is further configured to:

monitor the number of test media of the supply used in performing the analysis;

determine that a threshold value of test media has been reached, the threshold value being less than the quantity; and

perform, once the threshold value has been reached, an ordering procedure.

The monitoring may comprise receiving an indication of the quantity of the user's supply of test media.

The monitoring may comprise one of:

decrementing a value corresponding to the number of test media remaining in the user's supply when one or more test media is used; and

incrementing a value corresponding to the number of test media used from the user's supply when one or more test media is used.

The ordering procedure may comprise one or more selected from the group including automatically ordering test media, alerting a user to place an order, and presenting information necessary to order test media to a user.

### BRIEF DESCRIPTION OF THE DRAWINGS

For a better understanding of the embodiments and to show how it may be carried into effect, reference will now be made, purely by way of example, to the accompanying drawings.

With specific reference now to the drawings in detail, it is stressed that the particulars shown are by way of example and for purposes of illustrative discussion of selected embodiments only, and are presented in the cause of providing what is believed to be the most useful and readily understood description of the principles and conceptual aspects. In this regard, no attempt is made to show structural details in more detail than is necessary for a fundamental understanding; the description taken with the drawings making apparent to those skilled in the art how the several selected embodiments may be put into practice. In the accompanying drawings:

FIG. 1 is a schematic illustration of a system according to the presently disclosed subject matter;

FIG. 2 is a schematic illustration of a glucometer of the system illustrated in FIG. 1;

FIG. 3 is a schematic illustration of a remote computing system of the system illustrated in FIG. 1; and

FIG. 4 illustrates a method for ordering test media, for example using the system illustrated in FIG. 1, according to the presently disclosed subject matter.

### DETAILED DESCRIPTION OF THE INVENTION

As illustrated in FIG. 1, there is provided a system, which is generally indicated at 10, for measuring the glucose level of a user. The system 10 comprises a glucometer 12 and a remote computing device 14.

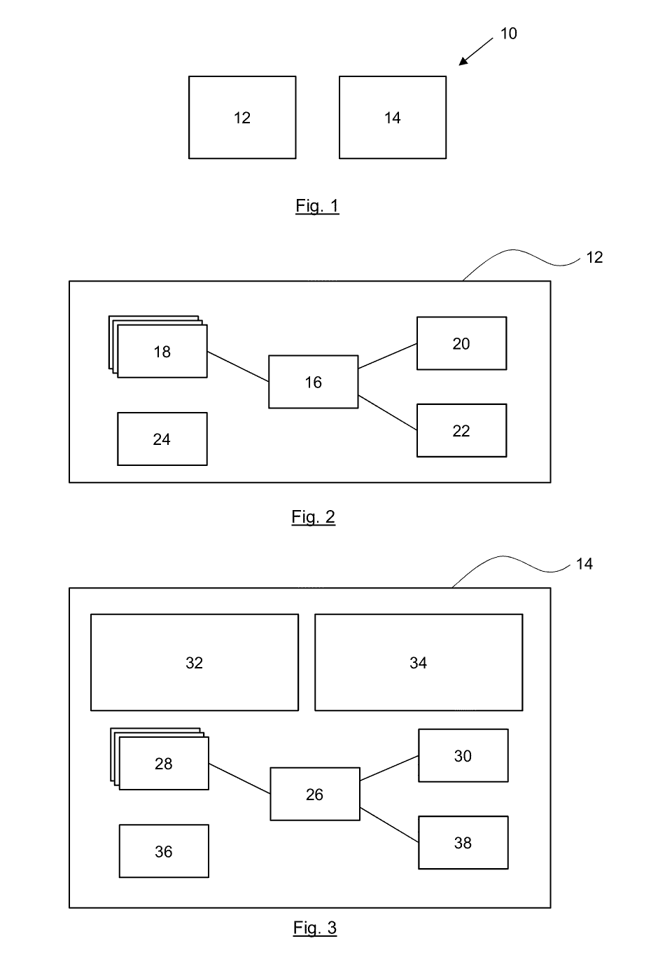

As illustrated figuratively in FIG. 2, the glucometer 12 comprises a processor 16, one or more memory modules 18 (which may comprise volatile and/or non-volatile memory), a media reader 20, a transmitter 22, and a power source 24. In addition, it may optionally comprise other elements (not illustrated), such as an external memory reader, a visual display such as an LCD or LED screen or LEDs, one or more ports configured for connection to a data cable, etc.

The media reader 20 is configured to facilitate analyzing a blood sample disposed on a test media (not illustrated), such as a test strip, disc, drum, cartridge, or any other suitable medium. It may be designed so as to facilitate detecting the glucose level in the blood sample using any suitable method. For example, in an electrochemical method, the blood sample reacts with one or more chemicals impregnated on the test medium. The amount of products of the reaction is proportional to the glucose level in the blood, and can be measured electrically by the media reader 20. Alternatively, the media reader 20 may operate using a coulometric or amperometric method, as is known in the art.

In addition, the media reader 20 may be configured to read information encoded on the test medium, including, but not limited to, calibration information, information regarding the make and/or model of the test medium, and information regarding the manufacturing of the test medium (such as batch number, manufacture date, expiration date, etc.).

Typically, the media is provided in packages having a known number of individual media therein.

The transmitter 22 is configured to transmit information regarding the results of the analysis to the remote computing device 14. For example, the glucometer 12 may be configured to transmit the information over a one-way communication channel, such as an audio-based channel, e.g., as described in co-pending U.S. application Ser. No. 14/071,744, and/or using a visual display. According to other examples, the glucometer 12 may be configured to transmit the information over a two-way communication channel, including, but not limited to, BLUETOOTH (i.e., one or more wireless technologies for exchanging data over short distances using short-wavelength radio transmissions in the ISM band from 2400-2480 MHz as per the standards defined by the Bluetooth Special Interest Group), NEAR FIELD COMMUNICATION (i.e., one or more technologies for smartphones and similar devices to establish radio communication with each other by touching them together or bringing them into close proximity, for example based on standards including, but not limited to, ISO/IES 18092 and those defined by the NFC Forum), WI-FI (i.e., one or more wireless local area network products that are based on the Institute of Electrical and Electonic Engineers' 802.11 standards), ZIGBEE (i.e., one or more of a suite of high level communication protocols used to create personal area networks built from small, low-power digital radios based on the Institute of Electrical and Electonic Engineers' 802.15 standard), and/or other suitable protocols.

According to any of the above examples, the transmitter 22 comprises the necessary hardware to facilitate transmission. For example, in the case wherein the glucometer 12 is configured to transmit information over an audio-based channel, the transmitter 22 comprises a suitable speaker. The glucometer 12 may optionally comprise a visual display, e.g., an LCD, LED, or other suitable screen or display, for example in a case wherein it is configured to transmit information using a visual display.

The transmitter 22 may be configured to transmit information over a wired and/or wireless indication channel, mutatis mutandis.

The processor 16 is configured to direct operation of the elements of the glucometer 12. As such, it may be configured to facilitate analysis of the blood sample, encode information for transmitting by the transmitter 22, direct operation of the transmitter, etc. In performing these tasks, it may utilize the one or more memory modules 18 for short-term storage of information.

The remote computing device 14 is any suitable device configured to receive information transmitted by the transmitter 22 of the glucometer 12, execute a program, display information to a user, and optionally receive commands from a user. It may also be configured to communicate with an external network, for example a public network such as the Internet, a POTS network, an ISDN network, cellular telephone system, and/or a VoIP system. As such, it may be any computing device, such as a mobile phone built on a mobile operating system (also referred to as a “smartphone”), a tablet computer, or any other suitable device. In particular, the remote computing device 14 is configured for installation thereon of third-party software.

As illustrated schematically in the FIG. 3, the remote computing device 14 comprises a processor 26, one or more memory modules 28 (which may comprise volatile and/or non-volatile memory), a receiver 30, a user-input interface 32, a user-output interface 34, and a power source 36. The user-input interface 32 and user-output interface 34 may be part of the same element, e.g., a touch-screen may constitute both.

In addition, the remote computing device 14 may comprise a transceiver 38, such as a modem and/or a wireless network adapter, configured to communicate with the external network. It will be appreciated that the receiver 30 may constitute part of the transceiver 38.

The processor 26 is configured to direct operation of the remote computing device 14. Inter alia, it is configured to execute software stored in the memory modules 28. In addition, the processor 26 may be configured to facilitate updating software stored in the memory modules 28, for example by downloading updated software from a remote server via the Internet.

When constituting part of the system 10, the remote computing device 14 is loaded with a software application which is configured to function with the glucometer 12. For example, the information transmitted by the glucometer 12 may contain raw data obtained by the media reader 20, which the software application is configured to interpret and provide a useful value based thereon. It may further be provided to track glucose levels over time, communicate with one or more outside servers, etc.

The system 10 as described above may be configured to monitor and/or track usage of test media by the glucometer 12. Software configured to perform the tracking may be installed in the memory of either the glucometer 12 or the remote computing device 14. Alternatively, the glucometer 12 and remote computing device 14 may each be loaded with complementary software which together perform this task.

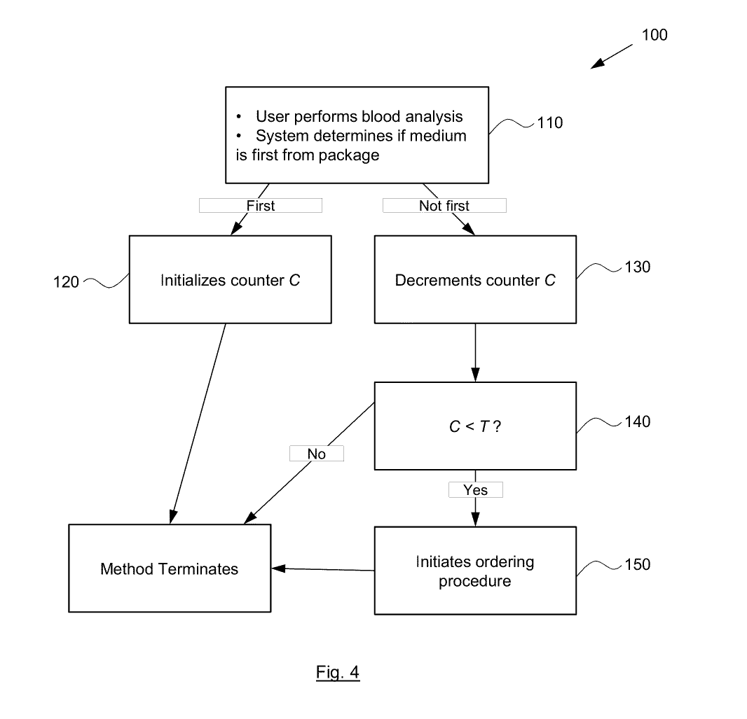

As illustrated in FIG. 4, the system 10, e.g., by running appropriate software (either installed on one of its constituent devices, or as complementary software on both), is configured to execute a method, which is generally indicated at 100. The method 100 may be executed in conjunction with the glucometer analyzing a test medium.

In step 110, a user utilizes the glucometer 12 to perform a blood analysis using a test medium, and the system 10 determines whether or not the medium being used for the analysis is the first one to be used from the package. According to some examples, the system 10 makes this determination based on user input, i.e., by prompting the user via the user-output interface 34 of the remote computing device 14, and/or by receiving an indication, which may be solicited (e.g., in response to a prompt) or unsolicited, as such by the user via the user-input interface 32.

According to other examples, the system 10 makes this determination automatically. For example, it may determine this based on the previous analysis being performed on the last test medium in its package. Alternatively, it may determine this based on information encoded on the test medium (e.g., the batch number) differing from that of the medium used in the previous analysis. According to any of the above examples, the system 10 may require that an automatic determination made thereby be confirmed by a user.

It will be appreciated that the system 10 may be configured to make the determination in one of several ways. For example, it may be configured to use one or more automatic methods to make the determination, but also be configured to receive an unsolicited indication from a user.

If the system 10 determines in step 110 that the test medium used in the analysis is the first one used from its package, then it proceeds to step 120 of the method, wherein it initializes a counter, which tracks the number of test media remaining in the package, as follows: C=N−1, where C is the value of the counter, and N is the number of test media initially in the package. According to some examples, the value of N may be determined by prompting a user, via the user-output interface 34, for the number of test media in the package. According to other examples, the system 10, e.g., via the remote computing device 14, may be configured to scan a barcode, such as a one-dimensional or two-dimensional (sometimes referred to as “matrix”) barcode, printed on the package, and associate information encoded therein with the quantity of test media within the package.

Following step 120, the method terminates until the next analysis of a test medium is performed.

If the system 10 determines in step 110 that the test medium used in the analysis is not the first one used from its package, it proceeds to step 130 of the method 100, wherein it decrements the counter by one, i.e., C=C−1. In the event that more than one test medium was used in the analysis, or if several analyses were performed before the system decremented the counter, the counter is decremented accordingly. For example, if n test media were used in an analysis, the counter would decrement as follows: C=C−n.

After decrementing the counter in step 130, the system 10 determines in step 140 whether or not a predetermined threshold value T has been passed, i.e., if the counter is lower than the threshold (C<7). The threshold value indicates at which quantity of remaining test media it is estimated that a new package should be ordered. The value may be based on the expected daily usage of test media, which may be provided by the user or determined by the system 10, for example based on usage history. In addition, the system 10 may take shipping times into account when determining the threshold value, and may further include a safety factor, for example determining the threshold value based on when an order should be placed to ensure that the user receive an ordered package several days before the current one is finished. Alternatively, the system 10 may allow a user to set a threshold value manually.

If the system 10 determines in step 140 that the threshold value has been passed, it proceeds to step 150 of the method, wherein it initiates an ordering procedure. The ordering procedure may be any procedure which is designed to facilitate or otherwise directly contribute toward a package of media being ordered.

According to some examples, the ordering procedure comprises the remote computing device 14 ordering one or more new packages of media via the Internet from an Internet-based merchant. This may be performed by the remote computing device 14 automatically. Accordingly, the remote computing device 14 and/or Internet-based merchant stores relevant information, e.g., shipping address, product to be ordered, etc. According to some modifications, the remote computing device 14 and/or Internet-based merchant may further store billing data, such as credit card information, billing address, etc. The remote computing device 14 may be configured to survey several ordering options for the best value. For example, it may check several Internet-based merchants for the best price for a particular product, it may check for the best unit price (i.e., cost per test medium) among several equivalent products, either at a single or at several Internet-based merchants, etc., and order from the one which provides the best value. In addition, in may be configured to take a preselected action if it identifies an unusually good value, for example as preset by the user. The preselected action may be one or more of, but is not limited to, ordering an increased quantity of packages and alerting the user that it identified the value. The user may preset what he considers to be a good value, for example more than a certain percentage lower per test medium than past purchases. The past purchases may be, e.g., an average or absolute best value of a predetermined number of last purchases, or an average or absolute best value of purchases made over a preselected amount of time.

According to other examples, the ordering may be a manual process, wherein the ordering procedure comprises alerting the user that the system 10 has determined that an order should be placed for a replacement package of test media. The remote computing device 14 may optionally present information and/or direction to facilitate the ordering by the user. For example, it may present on its user-output interface 34 one or more links, each redirecting a user to an Internet-based merchant which sells one or more packages of suitable test media.

According to further examples, some parts of the ordering may be automatic, as described above, with one or more manual steps. According to some modifications, the ordering procedure may comprise the remote computing device 14 automatically retrieving a website via which the user can purchase a package of test media. According to other modifications, the ordering procedure may comprise the remote computing device 14 retrieving all information necessary to place an order, e.g., product, price, merchant, estimate of shipping time, shipping address, billing address, billing information, etc., and presenting to the user for his approval, upon which the remote computing devices places the order accordingly.

The system 10 may utilize information regarding the ordering procedure in step 110, it may assume that the next package to be used is the one ordered, and initiate the counter C accordingly. Optionally, the system 10 may prompt the user to confirm the number of test media in the package the next time the counter C is initiallized.

Following step 150, the method terminates until the next analysis of a test medium is performed.

If the system 10 does not determine in step 140 that the threshold value has been passed, the method 100 terminates until the next analysis of a test medium is performed.

It will be appreciated that the above represents a basic method 100 which the system 10 is configured to follow to facilitate ordering of replacement test media by the system 10 based on usage of test media by the glucometer. The method 100 may be modified without departing from the spirit and scope of the presently disclosed subject matter, mutatis mutandis.

For example, the method as presented above may be modified, mutatis mutandis, to account for a user who orders more than one package of test media at a time, in which case it would be relevant to monitor the user's supply of test media, not just the media in one package. It will be appreciated that in the case wherein the user maintains a supply of one package, the quantity of test media in a single package is equal to the user's supply. Accordingly, the method 100 may be modified to add a second counter which monitors the number of complete packages of test media which have been used besides the number of test media remaining in the current package. For example, the counter C may be initialized to be the total number of test media the user purchases (for example, if the user orders 3 packages, each containing 50 test media, the value in step 110 of N would be 150). Alternatively, the method 100 may include an additional counter which monitors the number of packages of test media. Alternatively, the counter C may initially be set to the total supply of test media, for example by prompting the user for relevant information regarding his supply.

In addition or alternatively, the counter C may start at 0 or 1, and increment, with the threshold being passed in step 140 if the counter is higher than the threshold, i.e., C<T. Furthermore, the numerical operations presented herein may be shifted (e.g., in step 140, the system may determine that the threshold has been reached, i.e., that C=T; in step 120, the counter may be initialized as C=N; etc.).

Those skilled in the art to which this invention pertains will readily appreciate that numerous changes, variations and modifications can be made without departing from the scope of the invention mutatis mutandis.

Technical and scientific terms used herein should have the same meaning as commonly understood by one of ordinary skill in the art to which the disclosure pertains. Nevertheless, it is expected that during the life of a patent maturing from this application many relevant systems and methods will be developed. Accordingly, the scope of the terms such as computing unit, network, display, memory, server and the like are intended to include all such new technologies a priori.

As used herein the term “about” refers to at least ±10%.

The terms “comprises”, “comprising”, “includes”, “including”, “having” and their conjugates mean “including but not limited to” and indicate that the components listed are included, but not generally to the exclusion of other components. Such terms encompass the terms “consisting of” and “consisting essentially of”.

The phrase “consisting essentially of” means that the composition or method may include additional ingredients and/or steps, but only if the additional ingredients and/or steps do not materially alter the basic and novel characteristics of the composition or method.

As used herein, the singular form “a”, “an” and “the” may include plural references unless the context clearly dictates otherwise. For example, the term “a compound” or “at least one compound” may include a plurality of compounds, including mixtures thereof.

The word “exemplary” is used herein to mean “serving as an example, instance or illustration”. Any embodiment described as “exemplary” is not necessarily to be construed as preferred or advantageous over other embodiments or to exclude the incorporation of features from other embodiments.

The word “optionally” is used herein to mean “is provided in some embodiments and not provided in other embodiments”. Any particular embodiment of the disclosure may include a plurality of “optional” features unless such features conflict.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween. It should be understood, therefore, that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6 as well as non-integral intermediate values. This applies regardless of the breadth of the range.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable subcombination or as suitable in any other described embodiment of the disclosure. Certain features described in the context of various embodiments are not to be considered essential features of those embodiments, unless the embodiment is inoperative without those elements.

Although the disclosure has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the disclosure.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present disclosure. To the extent that section headings are used, they should not be construed as necessarily limiting.

What is claimed is:

1. A method of analyzing a blood sample, the method comprising:
providing a system configured to perform an analysis of a glucose level of the blood sample disposed on a test medium, wherein the test medium is one of a quantity of test media in a user's supply;
monitoring, by the system, the number of test media of the supply used in performing the analysis;
determining, by the system, that a threshold value of test media has been reached, the threshold value being less than the quantity; and
performing, by the system, once the threshold value has been reached, an ordering procedure.

2. The method according to claim 1, wherein the system comprises a glucometer configured to perform the analysis and transmit data, and a remote computing device configured to receive a transmission from the glucometer.

3. The method according to claim 2, wherein the remote computing device is configured to communicate with an external network.

4. The method according to claim 3, wherein the remote computing device is selected from the group including a mobile phone and a tablet computer.

5. The method according to claim 1, wherein the monitoring comprises receiving an indication of the quantity of the user's supply of test media.

6. The method according to claim 5, wherein the indication is at least partially based on information regarding a previous ordering procedure.

7. The method according to claim 1, wherein the monitoring comprises one of:
decrementing a value corresponding to the number of test media remaining in the user's supply when one or more test media is used; and
incrementing a value corresponding to the number of test media used from the user's supply when one or more test media is used.

8. The method according to claim 1, wherein the threshold value is determined at least partially based on expected daily usage of test media from the user's supply.

9. The method according to claim 8, wherein the expected daily usage of test media is at least partially based on usage history thereof.

10. The method according to claim 1, wherein the threshold value is determined at least partially based on expected shipping time of a new supply of test media.

11. The method according to claim 1, wherein the ordering procedure comprises the system automatically ordering test media.

12. The method according to claim 1, wherein the ordering procedure comprises the system alerting a user to place an order.

13. The method according to claim 1, wherein the ordering procedure comprises the system presenting information necessary to order test media to a user.

14. The method according to claim 13, further comprising the system ordering test media based on the information presented upon approval by a user.

15. The method according to claim 1, wherein the user's supply comprises a single package of test media.

16. The method according to claim 1, wherein the user's supply comprises more than one package of test media.

17. A system configured to perform an analysis of a glucose level of a blood sample disposed on a test medium, wherein the test medium is one of a quantity of test media in a user's supply, the system comprising:
a glucometer configured to perform the analysis and to transmit data; and
a remote computing device configured to receive a transmission from the glucometer and to communicate with an external network;
wherein the system is further configured to:
monitor the number of test media of the supply used in performing the analysis;
determine that a threshold value of test media has been reached, the threshold value being less than the quantity; and
perform, once the threshold value has been reached, an ordering procedure.

18. The system according to claim 17, wherein the monitoring comprises receiving an indication of the quantity of the user's supply of test media.

19. The system according to claim 17, wherein the monitoring comprises one of:
decrementing a value corresponding to the number of test media remaining in the user's supply when one or more test media is used; and
incrementing a value corresponding to the number of test media used from the user's supply when one or more test media is used.

20. The system according to claim 17, wherein the ordering procedure comprises one or more selected from the group including automatically ordering test media, alerting a user to place an order, and presenting information necessary to order test media to a user.
